(19) United States
(12) Patent Application Publication
(10) Pub. No.: US 2006/0148079 A1
(43) Pub. Date: Jul. 6, 2006

(54) Isolation and purification of human insulin producing cells for the treatment of insulin dependent diabetes

(76) Inventors: Virgil Paunescu, Timisoara (RO); Erika Iuliana Suciu, Timisoara (RO); Calin Adrian Tatu, Timisoara (RO); Tudor Ionel Oprea, Albuquerque, NM (US)

Correspondence Address: The Law Offices of Calvin B. Ward, Suite 305, 18 Crow Canyon Court, San Ramon, CA, 94583, US

(21) Appl. No.: 11/029,541

(22) Filed: Jan. 3, 2005

(51) Int. Cl. C12N 5/08

(52) U.S. Cl. 435/366

(57) ABSTRACT

A method for producing an ex vivo population of human insulin-producing cells that produce insulin in response to glucose from a patient is disclosed. The population of insulin producing cells is derived from human epithelial cells by causing those epithelial cells to differentiate into insulin producing cells. In one embodiment, the epithelial cells are derived from human mesenchymal stem cells (MSCs). The MSCs can be cultured from human bone marrow. In one embodiment, a culture of epithelial cell linage is generated by cultivating a sample of human cells containing a mixed culture containing human MSCs to produce an enriched culture of MSCs having a higher concentration of MSCs than the sample; and then differentiating cells from the enriched culture into cells of the epithelial cell lineage.

### BACKGROUND OF THE INVENTION

Insulin-dependent diabetes is a condition that affects tens of millions of people worldwide and is a significant cause of disabilities and death. The condition is the result of loss of the islet cells in the pancreas that produce insulin in response to the glucose levels in the blood. Individuals inflicted with this condition must control their blood sugar level manually by injecting themselves with insulin when their blood sugar levels rise. Inflicted individuals must test their blood sugar at regular intervals and take their injections whenever the sugar level is above a predetermined level. Failure to control their blood sugar adequately leads to numerous medical conditions including blindness and death.

The cost of the testing equipment, disposables, and the insulin is quite high. Hence, this condition also has a major financial impact on the inflicted individual both in terms of the cost of treatment and the inconvenience of the monitoring procedure. In addition, society as a whole is impacted financially both in terms of the portion of the cost that is born through various insurance funds and the lost of productivity of the inflicted individuals.

The possibility of treating this condition through the use of stem cells has been discussed for some time. In one such approach, stem cells are matured into insulin producing islet cells that are implanted in the patient. The cells, for example, can be periodically injected into the portal vein or under the kidney capsule to provide insulin in a manner more analogous to the control system used by a healthy body.

While this approach has promise, there are several problems. The first problem relates to obtaining suitable stem cells that can be matured into islet cells. While embryonic stem cells have been used in stem cell work, the large-scale creation of embryos for such work presents ethical problems that have hampered this line of work with human stem cells.

Second, rejection of the islet cells derived from the stem cells of one individual that are implanted in another individual remains a problem. While drugs for suppressing tissue rejection in transplant patients are known, the long-term treatment of the patient with such drugs poses a separate set of problems that are preferably avoided. Hence, a suitable source of stem cells from the individual to be treated is preferably utilized.

### SUMMARY OF THE INVENTION

The present invention includes an ex vivo population of human insulin-producing cells that produce insulin in response to glucose, a method for making the same, and a method for treating insulin-dependent diabetes using these insulin-producing cells. The population of insulin producing cells is derived from human epithelial cells by causing those epithelial cells to differentiate into insulin producing cells. In one embodiment, the epithelial cells are derived from human mesenchymal stem cells (MSCs). The MSCs can be cultured from human bone marrow. In one embodiment, a culture of epithelial cell lineage is generated by cultivating a sample of human cells containing a mixed culture containing human MSCs to produce an enriched culture of MSCs having a higher concentration of MSCs than the sample; and then differentiating cells from the enriched culture into cells of the epithelial cell lineage. In one embodiment, the cells of the epithelial lineage are caused to differentiate to the insulin-producing cells by culturing the cells of the epithelial lineage in a high-glucose media supplemented with a first mixture of growth and differentiation factors. In one embodiment, the first mixture includes epidermal growth factor, keratinocyte growth factor, hepatocyte growth factor, and vascular endothelial growth factor. In one embodiment, the first mixture further includes nicotinamide. The first mixture can also include gastrin, beta-cellulin, and forskolin. In one embodiment, human MSCs are cultivated in a vessel to which the human MSCs attach in a culture medium and includes a second mixture of growth and differentiation factors. In one embodiment, the second mixture of growth and differentiation factors includes epidermal growth factor, and keratinocyte growth factor. In one embodiment, the second mixture of growth and differentiation factors is replaced by a third mixture of growth and differentiating factors after a first incubation period. The third mixture can include beta-cellulin, hepatocyte growth factor, and vascular endothelial growth factor. The insulin-producing cells can be further grown in a culture medium that includes glucose supplemented with a mixture of growth factors that include epidermal growth factor, insulin-like growth factor-2, and nerve growth factor.

### BRIEF DESCRIPTION OF THE DRAWINGS

FIGS. 1 and 2 illustrate the manner in which the MSCs are purified and amplified in one embodiment of the present invention.

FIGS. 3 and 4 illustrate the differentiation of the MSCs into epithelial cells.

FIGS. 5 and 6 illustrate the differentiation of the epithelial cells to insulin-producing cells.

### DETAILED DESCRIPTION OF THE PREFERRED EMBODIMENTS OF THE INVENTION

The present invention utilizes a three-step approach to providing insulin islet cells for transplantation into the patient. In the first step, mesenchymal stem cells (MSCs) from the patient are purified and amplified in numbers. In the second step, the purified MSCs are differentiated into epithelial stem cells (ESCs). In the final step, the ESCs are differentiated into MSCs.

MSCs are pluripotential stem cells found in a number of locations including the bone marrow. The MSCs are capable of differentiating into any of the specific types of mesenchymal or connective tissues depending upon the growth conditions to which these cells are subjected. These cells are present at very low concentrations in the bone marrow, and hence, the MSCs must be isolated and substantially amplified by growth in vitro to provide a significant population of MSCs that can be utilized for the remaining steps of the procedure. It should be noted that the amplification growth step must not hamper the MSCs ability to differentiate to ESC in the subsequent steps of the procedure.

Techniques for extracting MSCs from human bone marrow and growing these extracted cells under conditions that result in a purified culture of MSCs are known to the art, and hence, will not be discussed in detail here. For example, U.S. Pat. No. 5,486,359, which is hereby incorporated by reference, describes a procedure for providing such a purified culture. For the purposes of the present discussion, it is sufficient to note that the isolation method utilizes the adherence of MSCs to plastic. The method starts from a tissue sample such as bone marrow containing the MSCs and to which is added a medium that contains various factors that stimulate MSC growth without differentiation. The cultured cells adhere to a substrate surface during this procedure, and hence, the culture is purified by removing the non-adhering cells. The growth medium is selected such that the only cells that can both grow in the medium and adhere to the surface are the MSCs.

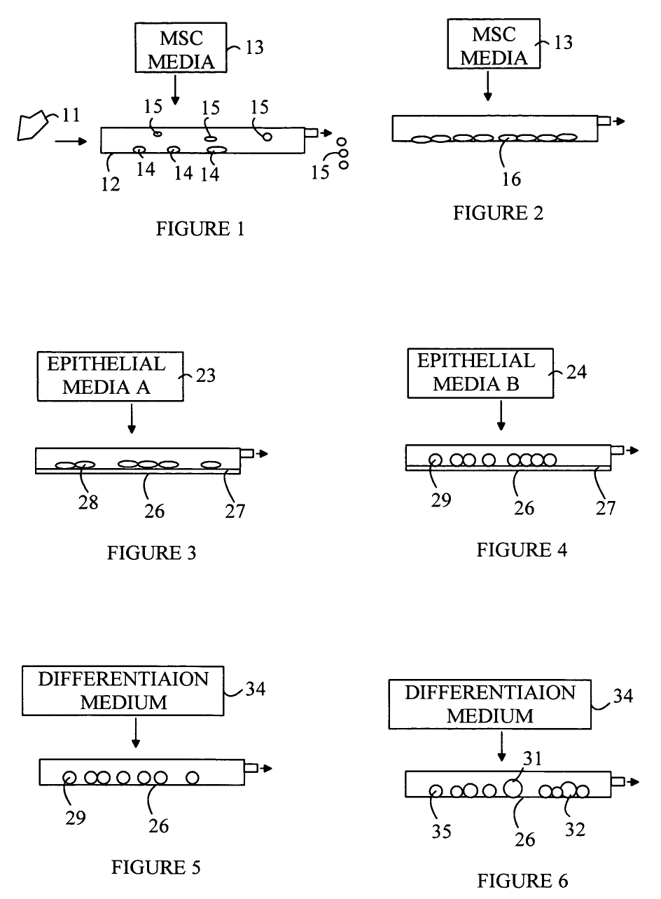

Refer now to FIGS. 1 and 2, which illustrate the manner in which the MSCs are purified and amplified in one embodiment of the present invention. A sample of bone marrow 11 is treated to isolate the fraction having MSCs and other cells that are present in the sample. Bone marrow is washed twice with DMEM medium to remove the heparin used in the collection of the marrow to prevent clotting. The washed marrow is then plated in flasks. After isolation, MSCs are cultivated in uncoated flasks 12 on DMEM low-glucose medium 13 (1 g/L) (Invitrogen Life Technologies, Carlsbad, Calif.), supplemented with 20% FBS (Hyclone, Logan, Utah) and 10 ng/ml FGF (Peprotech Inc., Rocky Hill, N.J.). The MSC cells 14 adhere to the flasks while the contaminating cells 15 do not and are washed from the flasks when the media is changed. The medium is changed every 3 days. After 14-21 days of plating, cells are replated on uncoated flasks, using the same culture media. After another 7 days period, when cells 16 reach 50 to 70% confluence as shown in FIG. 2, flow citometric analysis reveals a population 100% negative for CD45, CD34 (monoclonal antibodies, conjugated with PE or FITC are from Becton Dickinson, Franklin Lakes, N.J.), and AC133 (Miltenyi Biotec, Auburn, Calif.). These cells are self-regenerating cells, and are able to differentiate to osteoblasts, chondrocytes and adipocytes. If necessary, two passages are performed in order to obtain a 100% pure population of MSC.

In the second step, the MSCs are differentiated into cells of an epithelial lineage. Refer now to FIGS. 3 and 4, which illustrate the differentiation of the MSCs into epithelial cells. The human MSCs 27 obtained by the procedure discussed above are plated in flasks 26 coated with either gelatin or matrigel as shown at 27. The culture medium 23 is DMEM high-glucose (4,5 g/l) (Stemcell Technologies, Inc., St. Katharinen, Germany) with 20% FBS, and initially supplemented with 20-30 ng/ml epidermal growth factor (EGF) (Peprotech Inc., Rocky Hill, N.J.), and 10 ng/ml keratinocyte growth factor (KGF) (Peprotech Inc., Rocky Hill, N.J.). Referring to FIG. 4, after 3 days of culture, the media is changed to a second epithelial cell medium 24 by adding 1 nM beta-cellulin (R&D Systems, Minneapolis, Minn.), 10 ng/ml hepatocyte growth factor (HGF) (Peprotech Inc., Rocky Hill, N.J.), and 20 ng/ml vascular endothelial growth factor (VEGF) (Becton Dickinson, Franklin Lakes, N.J.). After 7-14 days from MSC plating, 30-50% of the cells 29 acquire a rounded, polygonal shape, and become positive for pancytokeratin.

Next, the epithelial cells derived from human MSCs by the procedure discussed above are differentiated into glucose-responsive insulin-producing beta pancreatic cells and islets. Refer now to FIGS. 5 and 6, which illustrate the differentiation of the epithelial cells to insulin-producing cells. Epithelial cells 29 are cultured in the DMEM high-glucose media 34, with 20% FBS, supplemented with 20-30 ng/ml epidermal growth factor (EGF), 10 ng/ml keratinocyte growth factor (KGF), 1 mM beta-cellulin, 10 mM hepatocyte growth factor (HGF), 20 ng/ml vascular endothelial growth factor (VEGF), 10 mM nicotinamide (Sigma, St. Louis, Mo.), 10 ng/ml gastrin (Sigma, St. Louis, Mo.) and 10 ng/ml ZnSO₄, in uncoated 50 ml Falcon tissue flasks (Becton Dickinson, Franklin Lakes, N.J.). After 2 to 3 days of culture, the cells are rounded and of various sizes. Both isolated cells 31 and 35 are grouped in islets 32, and become positively stained with dithizone (Sigma, St. Louis, Mo.) and anti-insulin antibodies (Dako Corporation, Carpinteria, Calif.). The fact that these cells respond to high glucose by producing insulin is also supported by the decrease of glucose concentration in the culture media over time.

It should be noted that the nicotinamide in this culture medium is toxic to any remaining MSCs that could contaminate the culture, and hence, selects for those epithelial cells that differentiate to the beta pancreatic cells.

The functionally mature insulin producing cells derived from human MSC can be grown and proliferated in the DMEM high-glucose medium with 20% FBS, supplemented with similar doses of epidermal growth factor (EGF), keratinocyte growth factor (KGF), beta-cellulin, hepatocyte growth factor (HGF), vascular endothelial growth factor (VEGF), nicotinamide and gastrin. In addition 60 ng/ml insulin-like growth factor-2 (IGF-2) (Peprotech Inc., Rocky Hill, N.J.), and 10 ng/ml nerve growth factor (NGF) (Peprotech Inc., Rocky Hill, N.J.) are introduced into the culture medium. The cellular composition includes, as a cellular population, 10-15% proliferating beta pancreatic cells and islets, which are glucose responsive insulin-secreting cells after 3-7 days. The beta pancreatic cells and islets obtained by differentiation from human MSC can be maintained and proliferated in culture for about 7-14 days, conserving their capacity to produce insulin. The supernatants collected from these cell cultures were analyzed for the presence of insulin, using a commercial ELISA kit (Diagnostic Systems Laboratories Inc., Webster, Tex.), the values obtained being between 200 and 500 μIU/ml.

The insulin-producing beta pancreatic cells obtained by the above-described procedure can be used to treat patients with insulin-dependent diabetes without the tissue rejection problems associated with pancreatic transplants. Such a treatment can be carried out by removing a sample of bone marrow from the patient and differentiating beta pancreatic cells ex vivo as described above. The beta pancreatic cells are then introduced into the patient. For example, the cells can be transplanted by injection into the portal vein.

Various modifications to the present invention will become apparent to those skilled in the art from the foregoing description and accompanying drawings. Accordingly, the present invention is to be limited solely by the scope of the following claims.

What is claimed is:

1. An ex vivo population of human insulin-producing cells that produce insulin in response to glucose, said population being derived from human epithelial cells.

2. The population of claim 1 wherein said epithelial cells are derived from human mesenchymal stem cells(MSCs).

3. The population of claim 1 wherein said MSCs are cultured from human bone marrow.

4. A method for producing a population of human insulin-producing cells comprising:
generating a culture of an epithelial cell lineage; and
causing said cells of said epithelial lineage to differentiate to human insulin-producing cells.

5. The method of claim 4 wherein said culture of epithelial cell lineage is generated by cultivating a sample of human cells containing a mixed culture containing human MSCs to produce an enriched culture of MSCs having a higher concentration of MSCs than said sample; and differentiating cells from said enriched culture into cells of said epithelial cell lineage.

6. The method of claim 5 wherein said cells of said epithelial lineage are caused to differentiate to said insulin-producing cells by culturing said cells of said epithelial lineage in a high-glucose media supplemented with a first mixture of growth and differentiation factors.

7. The method of claim 4 wherein said first mixture comprises: epidermal growth factor, keratinocyte growth factor, hepatocyte growth factor, and vascular endothelial growth factor.

8. The method of claim 7 wherein said first mixture further comprises nicotinamide.

9. The method of claim 7 wherein said first mixture further comprises gastrin, beta-cellulin, and forskolin.

10. The method of claim 5 wherein said human MSCs are cultivated in a vessel to which said human MSCs attach in a culture medium comprising a second mixture of growth and differentiation factors.

11. The method of claim 10 wherein said second mixture of growth and differentiation factors comprises epidermal growth factor, and keratinocyte growth factor.

12. The method of claim 10 wherein said second mixture of growth and differentiation factors is replaced by a third mixture of growth and differentiating factors after a first incubation period.

13. The method of claim 12 wherein said third mixture comprises beta-cellulin, hepatocyte growth factor, and vascular endothelial growth factor.

14. The method of claim 4 wherein said insulin-producing cells are further grown in a culture medium comprising glucose supplemented with a mixture of growth factors comprising epidermal growth factor, insulin-like growth factor-2, and nerve growth factor.

15. The method of claim 14 wherein said culture medium further comprises nicotinamide and gastrin.

16. A method for treating insulin dependent diabetes comprising:
generating cells of an epithelial cell lineage from a patient;
differentiating cells of said epithelial cell lineage to provide insulin producing cells that are responsive to the presence of glucose; and
transplanting said insulin producing cells into said patient.

17. The method of claim 16 wherein said cells of said epithelial cell lineage are generated by
removing a tissue sample containing MSCs from a patient;
culturing said sample to produce an enriched culture of MSCs;
differentiating MSCs from said enriched culture to provide cells of an epithelial cell lineage;

18. The method of claim 17 wherein said cells of said epithelial lineage are caused to differentiate to said insulin-producing cells by culturing said cells of said epithelial lineage in a high-glucose media supplemented with a first mixture of growth and differentiation factors.

19. The method of claim 18 wherein said first mixture comprises: epidermal growth factor, keratinocyte growth factor, hepatocyte growth factor, and vascular endothelial growth factor.

20. The method of claim 19 wherein said first mixture further comprises nicotinamide.

21. The method of claim 19 wherein said first mixture further comprises gastrin, beta-cellulin, and forskolin.

22. The method of claim 17 wherein said human MSCs are cultivated in a vessel to which said human MSCs attach in a culture medium comprising a second mixture of growth and differentiation factors.

23. The method of claim 22 wherein said second mixture of growth and differentiation factors comprises epidermal growth factor, and keratinocyte growth factor.

24. The method of claim 22 wherein said second mixture of growth and differentiation factors is replaced by a third mixture of growth and differentiating factors after a first incubation period.

25. The method of claim 24 wherein said third mixture comprises beta-cellulin, hepatocyte growth factor, and vascular endothelial growth factor.
